(19) United States
(12) Patent Application Publication
(10) Pub. No.: US 2021/0060034 A1
(43) Pub. Date: Mar. 4, 2021

(54) The Pharmaceutical Composition Intended to Inhibit HIV Infectivity in Order to Treat the Acquired Immune Deficiency Syndrome (AIDS) and Its Complications

(71) Applicant: Guy Faustin Monkam Nitcheu, Lille (FR)

(72) Inventor: Guy Faustin Monkam Nitcheu, Lille (FR)

(21) Appl. No.: 17/008,352

(22) Filed: Aug. 31, 2020

(30) Foreign Application Priority Data
Aug. 30, 2019 (FR) FR1909590

(51) Int. Cl. A61K 31/56; A61P 31/18

(52) U.S. Cl. CPC A61K 31/56; A61K 45/06; A61P 31/18

(57) ABSTRACT

This invention relates to a pharmaceutical composition used to inhibit pathogenic agents from penetrating target cells. It can also be used in the prevention or treatment of HIV infections, prevent the appearance or advance of AIDS and its various consequences.

This invention is concerned with a pharmaceutical composition that can be implied for therapeutic treatment of an infection by a pathogenic agent with a lipid bilayer envelope, in particular, the human immunodeficiency virus (HIV) and its complications.

### PRIOR ART

The AZT has been initially conceived in order to block the multiplication of cancerous cells and it has also been revealed to be inefficient and toxic at the same time. In return, its capacity to block one of the mechanisms of the VIH replication caused it to become the first medicine against acquired immune deficiency syndrome (AIDS). During previous centuries, some remarkable advancement has been made for prevention against this pandemic. This prevention means an integrated strategy that articulates the primary prevention with screening and care of seropositive people in a combined manner.

Moreover, the knowledge improvement with regard to infection process by the human immunodeficiency virus (HIV) has permitted the identification of new targets and the development of highly active anti-retroviral therapy (HAART). These treatments interrupt the HIV replication cycle and aim at reducing the quantity of virus circulating in the organism in a deep and sustainable manner and, in particular, to increase the number of “T” helper lymphocytes.

These anti-retrovirals are the synthetic molecules with different chemical nature. These have been subdivided into four big families: nucleosidic or non-nucleosidic reverse transcriptase inhibitors, protease inhibitors, fusion inhibitors and integrase inhibitors.
The nucleosidic reverse transcriptase inhibitors are the primary active developed antiretroviral on the HIV-1 and -2. These remain in competition with the natural substrates of reverse transcriptase and, thereby, block the pro-viral DNA production.
The non-nucleoside reverse transcriptase inhibitors (NNTRIs) act without interference with the enzyme's substrate. These are specific on the reverse transcriptase of the HIV-1 and inactive on the HIV-2. The protease inhibitors are peptidomimetics, these act at the assembly process level of newly synthesized protein virus. Therefore, by fixing themselves on active site of the viral protease, these prevent cleavage catalyzation of virus polypeptides. The protein maturation being altered as well as the virions formed remained inactive and non-infectious.
The fusion inhibitors prevent the HIV to fix itself to the external surface of cells and thus, to penetrate the latter.
The integrase inhibitors resists the integration of proviral DNA to genome of infected cell.

Therefore, the new strategies of treatment against HIV have appeared: These are the associations used for different purposes, whether these are lying within the context of combination of first line treatment, in this case, the triple therapy or more complex combinations are targeting the persons who have already been treated for accumulation of resistances and thereby justifying the passage to a quadri-therapy or penta-therapy. In relation to a immunotherapy by using AZT, these combined antiretroviral therapies have been found capable in order to efficiently remove the viral replication. Nevertheless, these only allow a partial regeneration of the immune system and the specific “T” immune response of the HIV has not been fully completely restored.

In addition, these antiviral therapeutic combinations never cure HIV infection, as well as, none of these vaccines have been developed based on viral or bacterial vectors, pseudo-virions or peptides protect against infection consecutive to an experimental test. Any type of treatment discontinuation is followed by a multiplication of viruses which remains in the body in latent state in “T” memory cells and the development of opportunistic diseases (tuberculosis, septicemia, fungal infections, parasitic infections, viral infections causing genital herpes, shingles, progressive multifocal leukoencephalitis) nature of the AIDS stages in seropositive patients. In addition, these drugs have been found to be responsible for significant side effects often transient but also for long-term. These include: physical disorders (body modifications, fatigue, bloating, nausea, vomiting, loss of appetite, fever, diarrhea, skin reactions . . . ), neuropsychic disorders (sleep disorders, feeling of emptiness, sadness, loss memory, cramps, muscle pain . . . ), impaired life quality (limitation due to fatigue, discomfort related to body modifications, neuropsychic disorders), hematologic abnormalities (anemia, lymphopenia, leukopenia, thrombocytopenia, neutropenia), more frequent hepatotoxicity in co-infected patients, localized or generalized lipodystrophy, dyslipidemia which may be associated with diabetes, cardiovascular disease, atherosclerosis, peripheral arteriopathy or peripheral arterial disease, thromboembolic diseases, kidney damage, bone abnormalities.

In addition, the prevalence of co-infection with hepatitis B virus (HBV) or hepatitis C virus (HCV) in people surviving with HIV is high because of similar infection patterns. These co-infections result in numerous recombinations between strains of virus or emerging mosaic strains known as circulating recombinant forms (CRF: Circulating recombinant forms). In addition, many HIV drugs have been found to be broken down by the liver, which can stress the body and cause more damage. The therapeutic management of HBV or HCV infection is limited and it considers the requirement to treat HIV infection or not, to integrate anti-HBV or anti-HCV and anti-HIV treatments into the heart of a global strategy in order to avoid chronicity of infection, to regress fibrosis and prevent complications of cirrhosis and prevalence of hepatocellular carcinoma. In effect, these different treatments interact with each other and may cause more serious side effects and thus impose virological, immunological, biochemical and histological surveillance constraints.

Moreover, the aging of patients surviving with HIV has led to emergence of co-morbidity and other causes of death, viz., cardiovascular diseases, cancers, especially cancers classifying AIDS (kaposi's sarcoma, lymphomas, cervix cancer, liver cancer, etc.), which present a new challenge in the care of persons who have been treated with anti-retrovirals in serological procedures. The HIV induced cellular immuno-depression would promote persistence and oncogenicity of pathogens. Moreover, the conventional cytotoxic chemotherapy targeting the tumor cells as well as the spinal cord line cells constitute a considerable risk on strongly depressed field.

In this context, the cell membrane have a key role in a large number of physiological or physiopathological processes and, in particular, in the infection. Indeed, it contains most of the essential elements for exchanges between the cell and its environment. It contains micro-domains known as rafts. The latter includes a compact assembly of lipids (glycosphingolipids and/or sphingomyelin and cholesterol) and proteins (glycosylphosphatidylinositol-anchored proteins, cholesterol related proteins, transmembrane proteins, double acetylated tyrosine kinases of Src family, the alpha subunits of heterotrimeric G proteins). The cholesterol playing a role as the dynamic “glue” that stiffens and structures these micro-domains. In particular, these proteins, like receptors such as insulin, IGF1, hybrid IR/IGF1R receptors, TLRs, co-receptors (CCR5, CXCR4) and many others show a determining role in cell signalization and which have been involved in certain inflammatory, infectious, metabolic disorders and carcinogenesis processes. Moreover, the dysfunction or the structural modifications which accompanies the activation of this membrane by pathogens or endogenous signals can be at the origin of an overproduction of micro-particles which contribute to replicate the inflammation, the systemic immune hyperactivation, the susceptibility coagulation, initiation, progression and aggravation of cardiovascular pathologies including atherosclerosis, myocardial infarction, stroke and cancer.

The viral inflammation explains a sizable part of the metabolic syndrome disorders, in particular, in patients who are infected with HIV. Not only, it would be linked to the accumulation of lipids in cells but also to the porosity of intestinal mucosa deteriorated by HIV which permits the passage of the intestinal lumen of bacteria into the bloodstream.

In the atherosclerotic process, the arterial inflammation is responsible for subendothelial accumulation of lipoproteins, especially cholesterol, which leads to an increase in release of pro-inflammatory cytokines, growth factors, increased expression of adhesion molecules, accumulation of apoptotic cells, and infiltration of macrophages which in turn produces pro-inflammatory mediators.

In chronic low-grade inflammation, the production of pro-inflammatory cytokines has been found to be involved in pathogenesis of type 2 diabetes.

Moreover, the inflammation is a symptom of autoimmune diseases which are characterized by a dysfunction of the immune system which is caused due to exposure on dangerous or toxic substances by a particular viral infection, by senescence of the immune cells or else by age.

In neurodegenerative diseases, in particular, in Alzheimer's disease, the inflammatory defense reactions caused by immune cells (macrophages, microglial cells, etc.) around these senile plaques would contribute to the disease progression.

These data raise the query of pharmacological targeting of cell membranes in the prevention and treatment of HIV infection as well for the diseases which have been characterized by chronic inflammation.

In addition, the beta-elemene is a broad-spectrum of anti-cancer properties. It has anti-inflammatory properties. Its immune-stimulatory properties are known. It improves the immunity and life quality of patients.

The d-limonene is regarded as its antiseptic, antiviral potential. It also shows antidiabetic and hypolipidemic properties and it may be considered for this purpose as a potential agent for prevention and treatment of metabolic disorders which are conventionally found in infectious diseases. Its anti-oxidant, anti-inflammatory and anticancer properties are well known in infectious diseases. In men, the d-limonene has also demonstrated low toxicity after a single and repeated dose for one year.

The beta-sitosterol and lupéol are the phytosterols which are having a molecular structure which is similar to cholesterol's structure. In this effect, these can compete with the latter. Their roles in plants are same as the cholesterol's role in humans, viz., in order to maintain the structure and function of the cell membrane. A number of scientific data have shown that these sterols have hypoglycemic, hypolipidemic, anti-inflammatory, antipyretic and anti-cancer properties. These also show anti-protozoan, broad-spectrum antimicrobial properties. Their pharmacological potential makes them therapeutic agents in metabolic syndrome disorders and opportunistic diseases at the AIDS stage.

The cinnamaldehyde is well considered for its hypoglycemic, hypolipidemic and anti-cancer properties. Its antibacterial, antifungal and antiviral properties are well known.

The allicin is well known for its anti-inflammatory, antioxidant, anticancer, antibacterial and antiviral properties. It helps in prevention of cardiovascular disease, in particular, by thinning the blood and fighting against excess cholesterol and high blood pressure episodes.

The ajoene, which is a derivative of allicin, is well known for its antioxidant, antithrombotic, anticancer properties in a broad spectrum (bactericidal and antifungal) and antiviral properties.

The kaempferol is known to have a good antioxidant property. It increases the stress of cancer cells during preservation of healthy cells. It is well known for modulation of a number of key elements in the signaling pathways which are involved in apoptosis, angiogenesis, inflammation and metastasis.

### TECHNICAL PROBLEM TO BE RESOLVED

An aim of this invention is to propose a new pharmaceutical composition which can be used as a medicament and, in particular, it can be used for treating the acquired immunodeficiency syndrome, adult immunodeficiency syndrome or severe combined immunodeficiency syndrome.

Another aim of the invention lies in proposition of a novel pharmaceutical composition which may be used as a medicament and, in particular, this may be used in the prevention and treatment of the acquired immunodeficiency syndrome which overcomes all or part of the disadvantages related to the compositions of the invention in the aforementioned prior art.

Another aim of this invention is to propose a pharmaceutical composition which decreases or inhibits the viral replication associated with a pro-inflammatory state of the cells.

Another aim of this invention is to propose a pharmaceutical composition which may be considered as a medicament for treatment and/or prevention of pathogenic infections.

Another aim of this invention is to propose a pharmaceutical composition which is well considered for inhibition of HIV entry into the target cells, in order to induce a neutralizing humeral immunity, a vigorous cellular immunity at the mucous membranes level, in particular, at the GALT level (Lymphoid tissues associated with the digestive tract).

Another aim of the invention is the proposition of a pharmaceutical composition which well considered for making it possible for blocking the infectivity of various wild viral strains even, in spite of their enormous genetic variability, as well as, for targetting the reservoir cells which carries the virus (latent forms) in the organisms patients followed by antiretroviral therapy.

Another aim of this invention is the reduction or even inhibition in enrichment of cells with cholesterol, the externalization of lipids, in particular, phosphatidylserine in the outer layer of the plasma membrane, and the formation of microparticles.

Another aim of this invention is to provide a pharmaceutical composition which destructures and restructures the lipid composition of the cell membrane and consequently leads to inhibition of the penetration of pathogen in the cells, the cellular signaling pathways is involved in viral replication, the secretion of pro-inflammatory cytokines, cell proliferation, angiogenesis and metastase.

Another aim of this invention lies in provision of a pharmaceutical composition which makes it possible to reduce or even inhibit the disorders of metabolic syndrome, in order to treat diabetes, dyslipidemias, cardiovascular diseases, cancer, cachectic syndrome, the aging process including senescence of immune cells.

Another object of this invention lies in provision of a pharmaceutical composition which can be used in the treatment and prevention of malignant tumors frequently associated with HIV-induced immunodepression, in particular, “classifying AIDS” cancers of immune cells, viz., the Kaposi's sarcoma, non-Hodgkin's malignant lymphoma (LMNH), cervical cancer and “classifying non-AIDS” cancers.

Another aim of this invention lies in provision of a pharmaceutical composition which may be used as a medicament, in particular, while treating a pathogen infection having a lipid bilayer envelope.

Another aim of this invention lies in provision of a pharmaceutical composition, in particular, as mentioned above, which has reduced toxicity and/or which is well tolerated by patients having a lipidic bilayer envelope.

### BRIEF DESCRIPTION OF THE INVENTION

In order to resolve at least one of the aforesaid technical problems, the present invention provides a pharmaceutical composition, which typically, in accordance with the invention, comprises as active ingredient a combination of beta-elemene, d-limonene, lupéol, of beta-sitosterol, cinnamaldehyde, allicin, ajoene and possibly kaempferol and its mixtures thereof.

The applicant has indeed found that such pharmaceutical composition was found to be active in the prevention and treatment of acquired immunodeficiency syndrome, including delaying the virus spread in body and reducing thus the number of opportunistic diseases as well as that their gravity.

The applicant has also demonstrated a synergistic effect of at least three of the constituents which provides a reinforced action of the composition on at least one mechanism involved in chronic inflammation, viz., a mechanism selected from the accumulation of lipids in the cells, micro-vesiculation of cell membranes, formation of microparticles, hyperactivation of cellular signaling pathways, overexpression of pro-inflammatory cytokines, autophagic degradation, penetration of pathogens into cells.

This pharmaceutical composition would combine the CD4+ receptor and/or the co-receptor(s) CCR5, CXCR4 and, thus, it prevents the formation of the gp120-CD4+-CCR5/CXCR4 complex and the real fusion between the viral and cellular membranes.

### DETAILED DESCRIPTION

The pharmaceutical composition, in accordance with the invention, may be used as a medicament and especially for its usage in the treatment of acquired immune-deficiency syndrome. In accordance with a particular embodiment of this invention, the composition of the invention may furthermore comprise a mixture of beta-sitosterol and cinnamaldehyde or a mixture of beta-sitosterol and kaempferol or a mixture of beta-sitosterol and allicin and/or ajoene.

As an example, it may be comprised in weight percentage of the total mass of the active ingredients, a percentage of beta-elemene being substantially equal to or greater than 10% and substantially equal to or less than 50%, and, in particular, substantially equal to or greater than 20% and substantially equal to or less than 30%, a mass percentage of d-limonene being substantially equal to or greater than 10% and being substantially equal to or less than 55%, in particular, being substantially equal to or greater than 20% and being substantially equal to or less than 40%, a mass percentage of lupéol or a pharmaceutically acceptable salt thereof being substantially equal to or greater than 10% and being substantially equal to or less than 50, and, in particular, being substantially equal to or greater than 20% and being substantially equal to or less than 30%, a percentage of sitosterol being substantially equal to or greater than 10% and being substantially equal to or less than 50%, and, in particular, being substantially equal to or greater than 20% and being substantially equal to or less than 30%, a percentage of cinnamaldehyde being substantially equal to or greater than 10% and being substantially equal to or less than 50%, and, in particular, being substantially equal to or greater than 20% and being substantially equal to or less than 40%, a percentage of allicin, when the said composition contains this ingredient, being substantially equal to or greater than 10% and being substantially equal to or less than 50%, and especially being substantially equal to or greater than 20% and being substantially equal to or less than 40%, a percentage of when said composition contains this ingredient, being substantially equal to or greater than 10% and being substantially equal to or less than 50%, and especially being substantially equal to or greater than 20% and being substantially equal to or less than 40%, a percentage of kaempferol when said composition contains this ingredient, being substantially equal to or greater than 10% and substantially equal to or less than 50%, and especially being substantially equal to or greater than 20% and being substantially equal to or less than 40%.

While the composition contains beta-elemene and d-limonene, their percentage by weight relative to the total mass of the active ingredients is, in particular, equal and, in particular, being substantially equal to 40%.

Furthermore, the composition, in accordance with the invention, comprises at least one pharmaceutically acceptable excipient. This excipient can be solid or liquid. It may be selected, for example, from purified water, ethyl alcohol, propylene glycol, glycerine, vegetable oils, animal oils, hydrocarbons, silicones, sugars such as glucose, levulose, wheat starch, corn starch, potato starch, xanthan gum, gum arabic, gum tragacanth, gum Sterculia, guar gum or “guaranates”, pectins, alginates, carrageenates, agar or Agar-Agar, gelatin, cellulose and its derivatives.

The composition of invention can be administered by any suitable way, viz., by oral, rectal, local (topical, for example), intraperitoneal, systemic, intravenous, intramuscular, subcutaneous or mucosal way, especially sublingual or by using a patch, or in a form encapsulated in, or immobilized on, liposomes, microparticles, microcapsules, associated with nanoparticles and the like. These include, by way of nonlimiting examples of excipients suitable for oral administration, talc, lactose, starch and its derivatives, cellulose and its derivatives, polyethylene glycols, polymers of acrylic acid, gelatin, magnesium stearate, animal, vegetable or synthetic fats, paraffin derivatives, glycols, stabilizers, preservatives, antioxidants, wetting agents, anti-caking agents, dispersants, emulsifiers, taste modifying agents, penetration agents, solubilization agents. The formulation techniques and administration of drugs and pharmaceutical compositions are well known in the art which is considered here, the skilled person may, in particular, refer to the book Remington's Pharmaceutical Sciences, latest edition and the like.

In accordance with the invention, the composition can be advantageously administered orally or by intravenous injection.

As an advantage, the composition, in accordance with the invention, is adapted to be administered orally or intravenously at a dose quantity being substantially equal to or greater than 40 mg/kg/24 h and substantially equal to or less than 200 mg/kg/24 h in one or more doses, which a mammal with such a requirement.

The composition, in accordance with the invention, may advantageously be used in the prevention and/or treatment of infections with a pathogen including a lipid bilayer envelope, and, in particular, HIV, in the prevention and/or treatment of the acquired immuno-deficiency syndrome, opportunistic diseases, including candidal infections, tuberculosis, cytomegalovirus infections (CMV), in cryptosporidiosis, isosporidiosis, atypical mycobacteria, recurrent salmonella non-thyphi septicemia, chronic ulcers, bronchial, pulmonary or oesophageal infections, disseminated or extrapulmonary coccidioidomycosis, disseminated or extra-pulmonary histoplasmosis, cryptococcosis, extra pulmonary pneumocystis carinii pneumonia, progressive multifocal leukoencephalopathy, cerebral toxoplasmosis.

As an example, the composition, in accordance with the invention, can be used for reduction or eradication of the reservoir of pathogens in the digestive tract, the numerous immunological and architectural abnormalities during infection with pathogens, particularly the HIV, for restoration of competent mucosal immunity, especially T-cells.

The mode of action of the invention composition has not been fully elucidated, in particular, the “T” lymphocytes. It is probable that it acts simultaneously on different mechanisms by destructuring and restructuring the lipid composition of cells, infectious agents, especially membrane, by changing the conformation of the proteins that are existing there, and thus in prevention of the action binding proteins, input cofactors, alternative receptors or alternative co-receptors, adsorption, endocytosis, ultimately penetration of pathogens into cells and activation of cell signaling pathways which are involved in many pathophysiological processes.

This membrane lipid composition modification could also reduce or even inhibit the affinity, assembly, budding and maturation of viral particles (certain viral proteins such as nef, vif, vpr) at the rafts' level or membrane micro-domains' level.

On the other hand, this composition, in accordance with the invention, could also be used in the prevention and/or treatment of infections by non-enveloped membrane viruses.

Thus, the composition, in accordance with the invention, would inhibit the formation, overexpression and/or dysfunction of certain sphingolipids, in particular, glycosphingolipids, membrane microvesicles, overexpression of chemokine receptors. (CXCR4 and/or CCR5), secretion of pro-inflammatory cytokines (TNF alpha, IL-lb, IL-6, IL-8), viral replication, chronic inflammation and systemic immune hyperactivation, it would lead to preservation of competent mucosal immunity, especially HIV-targeted immune cells, particularly CD4+ lymphocytes, decrease or inhibit abnormalities in the architecture of lymphoid tissue associated with the gastrointestinal tract (GALT).

Moreover, it can be also used in the prevention and/or treatment of disorders of the metabolic syndrome, cardiovascular diseases, stroke, peripheral arterial diseases, deep vein thromboses, pulmonary diseases, including pulmonary embolism, autoimmune diseases, hepatic, renal, neurodegenerative diseases, complications in the central nervous system (neuroAIDS).

In addition, this composition could also be used in the prevention and/or treatment of cancers, including cancers classifying AIDS selected from Kaposi's sarcoma, burkitt lymphoma, immunoblastic lymphoma, primary cerebral lymphoma, non-Hodgkin's malignant lymphoma (NHLH).), and non-classifying AIDS cancers selected from oral cancer, stomach cancer, colon cancer, in particular, invasive colon or colorectal cancer, rectal cancer, anal cancer, liver cancer, hepatocellular carcinoma, gall bladder cancer, pancreatic cancer, lung cancer, especially lung adenocarcinoma, chronic or acute leukemia, multiple myeloma, Hodgkin lymphoma, tumors of the brain and others to localization in the nervous system, bladder cancer, ovarian cancer, uterine cancer, kidney cancer, prostate cancer and breast cancer, bone tumors.

For this, it inhibits cell proliferation, angiogenesis and metastasis and causes apoptosis of cancer cells. It would also lead to reduction of inflammatory conditions and thus consolidate a favorable ground for optimizing health.

This invention also relates to a pharmaceutical preparation which comprises the composition, in accordance with the invention, and, in addition, as a mixture or separately packaged, at least one anti-diabetic and/or hypolipidemic agent, an anti-cancer agent, an immunostimulatory agent, as an antiviral agent for their usage in the therapeutic treatment of acquired immunodeficiency syndrome, tumor pathology and disorders of the metabolic syndrome, simultaneously, sequenced or spaced apart over time therefore a favorable ground for health optimization.

For example, the antidiabetic agent may be selected from biguanides, sulphonamides hypoglycémiants and glinides, alpha-glucosidase inhibitors, incretins including GLP-1, insulin, lipid-lowering agent may be selected from statins, fibrates, ezetimibe, nicotinic acid, cholestyramine, the anti-cancer agent is selected from the anti-metabolites (methotrexate, capecitabine, 5-fluorouracil), the alkylating agents (cisplatin, mitomycin c, busulfan), the molecules implying an action on mitotic spindle (vinblastine, vincristine, doxetaxel), the tyrosine kinase inhibitors (afatinib, erlotinib, sunitinib), the serine threonine kinase inhibitors (vemurafenib, everolimus, temsirolimus), the agents acting on topo isomerase (daunorubicin, doxorubicin, etoposide), proteasome inhibitors, DNA methyltransferase inhibitors, histone deacetylase inhibitors, immunomodulators (interferons, corticosteroids, talimogen), monoclonal antibodies (cetuximab, gemtuzumab, rituximab), certain genetically modified viruses which preferentially target cancer cells, curcumin, glutathione, vitamin C and its mixtures thereof, and, in particular, a mixture of two of the said anti-cancer agents, the radioactive agents which can be used in brachytherapy and/or the injectable or ingestible active metabolites, the antiviral agent may be selected from abacavir, abacavir+amphiprin, abacavir+lamivudine+zidovudine, amprenavir, atazanavir, AZT, capravirine, darunavir, ddc, ddl, ddl (enteric coating), delavirdine, dolutegravir, doravirine, efavirenz, efavirenz+emtricitabine+tenofovir DF, 4-ethynyl-2-fluoro-2′-deoxyadenosine, elvitegravir, emtricitabine+tenofovir DF, emvirine, enfuvirtide, enteric coating based on didanosine, etravirine, fosamprenavir calcium, indinavir, lamivudine, lamivudine+zidovudine, lopinavir, lopinavir+ritonavir, maraviroc, nelfinavir, nevirapine, PPL-100, raltegravir, rilpivirine, ritonavir, saquinavir, stavudine, tipranavir or vicriviroc.

This invention also relates to a pharmaceutical preparation which comprises in combination beta-elemene and/or d-limonene, lupéol and/or beta-sitosterol, allicin and/or ajoene, cinnamaldehyde and optionally kaempferol.

Thus, this invention relates to a pharmaceutical preparation which comprises in combination beta-elemene and/or d-limonene, lupéol or a pharmaceutically acceptable salt thereof and/or beta-sitosterol, allicin and/or ajoene, cinnamaldehyde and possibly kaempferol.

Thus, this invention relates to a dietary supplement which comprises in combination beta-elemene and/or d-limonene, lupéol and/or beta-sitosterol, allicin and/or ajoene, cinnamaldehyde and optionally kaempferol.

### DEFINITIONS

The term “therapeutic treatment” refers to curative treatment and prophylactic treatment; within the meaning of this invention, a therapeutic treatment makes it possible to at least partially restore, at least partially correct or at least partially modify the physiological functions by exerting a pharmacological, immunological or metabolic action.

The term “inflammation” refers to a reaction of the body's stereotyped immune system to external aggression (infection, trauma, burn, allergy, etc.) or internal (cancerous cells). The chronic nature of this inflammation is intended to maintain the (or the) aggression factor.

The term “patient” refers to an animal or human mammal. The composition, in accordance with the invention, can also be used in veterinary medicine.

For the purpose of this invention, an “anti-cancer agent” is an element which has, at least in vitro, an action against cancer cells, regardless of its mechanism of action. For the purposes of this invention, the term “action” means the destruction or at least partial modification of the cancer cells, thus, making it possible, in particular, to limit the proliferation of the cancer cells and/or their propagation.

The term “viral infection” refers to a biological entity whether it is the hepatitis B virus (HBV), the hepatitis C virus (HCV), the human immunodeficiency virus (HIV), the virus herpes (HSV, herpes simplex virus), the cytomegalovirus (CMV), requiring a host, usually a cell, which uses the constituents to be multiplied.

The term “opportunistic disease”, in human or veterinary medicine, refers to a disease caused by usually mildly aggressive germs that may cause serious complications by affecting people with weakened immune systems, such as those undergoing transplantation, organ, chemotherapy or suffering from AIDS.

The term “patients with diabetes”, refers to patients surviving with type 1 diabetes, patients surviving with type 2 diabetes, patients surviving with gestational diabetes, patients surviving with diabetes insipidus and patients surviving with renal diabetes.

The term “dyslipidemia”, refers to hyperlipidemia and hypolipidemia, which has been determined, in accordance with criteria, in effect.

For this invention, a “dietary supplement”, is a foodstuff which is intended to supplement the normal diet and which constitutes a concentrated source of nutrients or other substances having a alone or combined nutritional or physiological effect.

With regard to the above-mentioned agents, the terms includes, unless otherwise specified, the isomers of constitution, stereoisomers of conformation, enantiomers and diastereoisomers of the chemical compound considered.

With regard to the d-limonene in composition, in accordance with the invention, the term includes, unless otherwise specified, its derivatives, in particular, perillic acid, dihydroperillic acid, perilyl alcohol, limonene 1,2-diol and its mixtures thereof.

With regard to lupéol in the composition, in accordance with the invention, the term includes, unless otherwise specified, a pharmaceutically acceptable salt thereof (lupéol acetate, lupéol toluate, lupéol palmitate, lupéol steroid, lupéol cinnamate, lupéol-m). chlorobenzoate, lupéol-p-chlorobenzoate), its derivatives, in particular, stigmasterol, campesterol, betulinol, betulinic acid, 3-O-(3′,3′-dimethylsuccinyl) -betulinic acid, the acid 7β-(4-hydroxybenzoyloxy) -betulinic and their mixtures thereof.

With regard to cinnamaldehyde (CA) in the composition, in accordance with the invention, the term includes, unless otherwise specified, its derivatives, in particular, hydroxycinnamic acid, 2-hydroxycinnamaldehyde (HCA), 2′-benzoyloxycinnalmaldehyde (BCA), cinnamtannin B1, formation dimers, especially HCA-HCA, BCA-BCA, CA-CA and their mixtures thereof.

With regard to the allicin in the composition, in accordance with the invention, the term includes unless otherwise specified, alliine, its derivatives including diallyl sulfide, diallyl disulfide, diallyl trisulfide, diallyl tetrasulfide, trisulphide methyl, methyl allyl sulfide, ajoene and their mixtures thereof.

Regarding kaempferol in the composition, in accordance with the invention, the term includes, unless otherwise specified, kaempferol-3, 7-O-α-1-dirhamnoside and its mixtures thereof.

### SOME EXAMPLES

The percentage of the compositions below is a percentage by weight relative to the total mass of the active ingredients. Non-exhaustive examples: Composition 1a: d-limonene (20%), lupéol (20%), b-sitosterol (20%), cinnamaldehyde (20%) and allicin (20%).

Composition 1b: beta-elemene (20%), lupéol (20%), b-sitosterol (20%), cinnamaldehyde (20%) and allicin (20%).

Composition 2: d-limonene (20%), beta-elemene (20%), lupéol (20%), b-sitosterol (20%), cinnamaldehyde (10%) and allicin (10%).

Composition 3a: d-limonene (30%), lupéol (30%), b-sitosterol (10%), cinnamaldehyde (10%) and allicin (20%).

Composition 3b: beta-elemene (30%), lupéol (30%), b-sitosterol (10%), cinnamaldeh_{y}de (10%) and allicin (20%).

Composition 4a: d-limonene (20%), lupéol (30%), b-sitostérol (20%), cinnamaldehyde (20%) and allicin (10%).

Composition 4b: beta-elemene (20%), lupéol (30%), b-sitosterol (20%), cinnamaldehyde (20%) and allicin (10%).

Composition 5a: d-limonene (10%), lupéol (10%), b-sitostrol (20%), cinnamaldehyde (20%) of allicin (20%) and ajoene (20%).

Composition 5b: beta-elemene (10%), lupéol (10%), b-sitosterol (20%), cinnamaldehyde (30%), allicine (20%) and ajoene (20%).

Composition 6a: d-limonene (20%), beta-elemene (20%), lupéol (10%), b-sitosterol (20%), cinnamaldehyde (20%) and allicin (10%).

Composition 6b: d-limonene (20%), beta-eleméne (20%), lupéol (10%), b-sitosterol (20%), cinnamaldéhyde (20%) and ajoene (10%).

Composition 7a: d-limonene (10%), lupéol (20%), b-sitosterol (30%), cinnamaldëhyde (30%) and allicine (10%).

Composition 7b: beta-elemene (10%), lupéol (20%), b-sitostër (30%), cinnamaldehyde (30%) and allicin (10%).

### EXPERIMENTAL RESULTS

In order to examine the antiviral activity of this pharmaceutical composition, the peripheral blood mononuclear cells (PBMCs) were activated by a mitogen, phytohemagglutinin and then infected with lymphocytic tropism strains. All these cells were maintained at 37° C. under 5% CO2 in RPMI 1640 containing 10% (vol/vol) fetal calf serum, 2 mM of 1-glutamine, a solution of three antibiotics (penicillin, streptomycin, neomycin). Seven days after its infection, the culture supernatants have been collected and frozen at −20° C. until the measurement of viral replication by quantification of the enzyme activity of the reverse transcriptase.

In parallel, on the seventh day of the culture, the cytotoxicity of the molecules had been evaluated with respect to PBMC activated by phytohemagglutinin, but not found to be infected. These possible effects on cell viability were measured by using a colorimetric test with methyl tetrazolium salt.

The percentage of inhibition of reverse transcriptase enzymatic activity and cell viability will be calculated in order to determine effector concentrations 50, 70 and 90% (EC50, CE70 and CE90) and cytotoxic concentrations (CC50, CC70 and CC90) from a four-parameter dose-effect curve. The selectivity indices (SI) were calculated In accordance with the ratio CC50/EC50.

Therefore, in the compositions to be tested, a decrease in the binding of the virus to PBMCs has been observed, a limitation of viral replication. The mode of action against HIV could be attributed to the inhibition of early events of viral replication, in particular, the adsorption of the virus. This pharmaceutical composition could also inhibit the chronic inflammation seen in people living with HIV.

Therefore, this pharmaceutical composition can be used rightly in the prevention and treatment of acquired immunodeficiency syndrome, opportunistic diseases related to immunodeficiency, disorders of the metabolic syndrome and cancer.

1. A pharmaceutical composition characterised by its consisting of a combination of beta-elemene, d-limonene, lupéol or one of its pharmaceutically acceptable salts, beta-sitosterol, allicin, ajoene, cinnamaldehyde and optionally kaempferol.

2. The pharmaceutical composition of claim 1, characterised by its consisting of, additionally, a mixture of beta-sitosterol and cinnamaldehyde or of beta-sitosterol and allicin and/or ajoene or a mixture of beta-sitosterol and kaempferol.

3. The pharmaceutical composition of claim 1, characterised by its consisting of the following percentages by mass of the total mass of active ingredients, a percentage of beta-elemene substantially equal to or greater than 10% and substantially equal to or less than 50%, and in particular substantially equal to or greater than 20% and substantially equal to or less than 30%, a percentage by mass for d-limonene that is substantially equal to or greater than 10% and substantially equal to or less than 55%, in particular substantially equal to or greater than 20% and substantially equal to or less then 40%, a percentage of lupéol or one of its pharmaceutically acceptable salts that is substantially equal to or greater than 10% and substantially equal to or less than 50%, and in particular substantially equal to or greater than 20% and substantially equal to or less than 30%, a percentage of beta-sitosterol that is substantially equal to or greater than 10% and substantially equal to or less than 50%, and in particular substantially equal to or greater than 20% and substantially equal to or less than 30%, a percentage of cinnamaldehyde that is substantially equal to or greater than 10% and substantially equal to or less than 50%, and in particular substantially equal to or greater than 20% and substantially equal to or less than 40%, a percentage of allicin, when the composition contains this ingredient, that is substantially equal to or greater than 10% and substantially equal to or less than 50%, and in particular substantially equal to or greater than 20% and substantially equal to or less than 40%, a percentage of ajoene, when the composition contains this ingredient, substantially equal to or greater than 10% and substantially equal to or less than 50%, and in particular substantially equal to or greater than 20% and substantially equal to or less than 40%, a percentage of kaempferol, when the composition contains this ingredient, that is substantially equal to or greater than 10% and substantially equal to or less than 50%, and in particular substantially equal to or greater than 20% and substantially equal to or less than 40%.

4. The pharmaceutical composition of claim 1, used as medication, and in particular used to reduce or eradicate a build-up of pathogenic agents within the digestive tract, various immunological and architectural anomalies during infection by pathogenic agents and in particular HIV, to restore competent mucosal immunity, in particular T lymphocytes.

5. The pharmaceutical composition of claim 4, for use in the prevention and/or treatment of chronic inflammation, immune system hyperactivity and cachexia.

6. The pharmaceutical composition of claim 1, for use in the prevention and/or treatment of infections, in particular HW, in the prevention and or treatment of acquired immunodeficiency syndrome, opportunistic diseases, in particular candida infections, tuberculosis, cytomegalovirus infections (CMV), cryptosporidiosis, isosporidiosis, atypical mycobacteria, septicemia caused by recurrent non-thyphi salmonella, chronic ulcers, bronchial, pulmonary or oesophageal infections, disseminated or extra-pulmonary coccidioidomycosis, disseminated or extra-pulmonary histoplasmosis, extra-pulmonary cryptococcosis, pneumonia caused by Pneumocystis carinii, progressive multifocal leukoencephalopathy and cerebral toxoplasmosis.

7. The pharmaceutical composition of claim 1, for use in the prevention and/or treatment of diabetes, complications arising from metabolic syndrome, cardiovascular diseases, stroke, diseases of the peripheral arteries, deep vein thrombosis, pulmonary diseases, in particular pulmonary embolism, auto-immune, liver or renal diseases, neurodegenerative diseases, complications within the central nervous system (neuroAIDS).

8. The pharmaceutical composition of claim 1, for use in the prevention and/or treatment of cancer, in particular forms of cancer classified as AIDS from Kaposi's sarcoma, Burkitt lymphoma, immunoblastic lymphoma, primitive cerebral lymphoma, non-Hodgkin malignant lymphoma (NHML) and forms of cancer not classified as AIDS from mouth cancer, stomach cancer, colon cancer, in particular invasive colon or colorectal cancer, rectal cancer, anal cancer, liver cancer, hepatocellular carcinoma, cancer of the gallbladder, pancreatic cancer, lung cancer, in particular adenocarcinoma of the lung, chronic or acute leukemia, multiple myeloma, Hodgkin lymphoma, brain tumours and others located within the nervous system, cancer of the bladder, ovarian cancer, uterine cancer, kidney cancer, prostate cancer and breast cancer and bone tumours

9. A pharmaceutical preparation characterised by its consisting of the composition of claim 1 and, in addition, mixed in or packaged separately, at least one anti-diabetic and/or hypolipidemic agent, one anti-cancer agent, one immunostimulant agent, one antiviral agent for use in the treatment of acquired immunodeficiency syndrome, tumour pathology or complications arising from metabolic syndrome.

10. The pharmaceutical preparation of claim 9, characterised in that the said anti-diabetic agent may be selected from biguanides, sulphonylureas and glinides, alpha-glucosidase inhibitors, incretins including GLP-1, insulin, the hypolipidemic agent may be selected from statins, fibrates, Ezetimibe, nicotinic acid, colestyramine, the anti-cancer agent is selected from anti-metabolites (methotrexate, capecitabine, 5-fluorouracil), alkylating agents (cisplatin, mitomycin c, busulfan), molecules that act on the mitotic spindle (vinblastine, vincristine, docetaxel), tyrosine kinase inhibitors (afatinib, erlotinib, sunitinib), serine threonine kinase inhibitors (vemurafenib, everolimus, temsirolimus), agents that act on topo-isomerase (daunorubicin, doxorubicin, etoposide), proteasome inhibitors, DNA methyltransferase inhibitors, histone deacetylase inhibitors, immunomodulators (interferons, corticosteroids, talimogene), monoclonal antibodies (cetuximab, gemtuzumab, rituximab), some genetically modified viruses that target cancerous cells, curcumin, glutathione, vitamin c and their mixtures, and in particular the mixture of the two stated anti-cancer agents, radioactive agents that can be used in brachytherapy and/or injectable or ingestible active metabolites, the antiviral agent can be selected from abacavir, abacavir+amphiprine, abacavir+lamivudine+zidovudine, amprenavir, atazanavir, AZT, capravirine, darunavir, ddc, ddl, ddl (enterosoluble coating), delavirdine, dolutegravir, doravirine, efavirenz, efavirenz+emtricitabine+tenofovir DF, 4-ethynyl-2-fluoro-2′-desoxyadenosine, elvitegravir, emtricitabine+tenofovir DF, emvirine, enfuvirtide, didanosine base enteriquea coating, etravirine, fosamprenavir calcium, indinavir, lamivudine, lamivudine+zidovudine, lopinavir, lopinavir+ritonavir, maraviroc, nelfinavir, nevirapine, PPL-100, raltegravir, rilpivirine, ritonavir, saquinavir, stavudine, tipranavir or vicriviroc.
